(19) United States
(12) Patent Application Publication
(10) Pub. No.: US 2016/0113571 A1
(43) Pub. Date: Apr. 28, 2016

(54) ACTIVE MEDICAL DEVICE FOR THE SELECTIVE AND EARLY TREATMENT OF HYPOPNEAS

(71) Applicants: SORIN CRM SAS, Clamart (FR); INSERM - INSTITUT DE LA RECHERCHE MÉDICALE, Paris (FR); UNIVERSITÉ DE RENNES 1, Rennes (FR); UNIVERSITÉ JOSEPH FOURIER, Grenoble Cedex 9 (FR)

(72) Inventors: Amel Amblard, Sceaux (FR); Laurence Graindorge, Thouare sur Loire (FR); Delphine Feuerstein, Issy les Moulineaux (FR); Alfredo Hernandez, Cesson Sevigne (FR); Jean-Louis Pépin, Grenoble (FR)

(21) Appl. No.: 14/922,539

(22) Filed: Oct. 26, 2015

(30) Foreign Application Priority Data
Oct. 27, 2014 (FR) 1460320

(51) Int. Cl. A61B 5/00; A61B 5/0488; A61B 5/0476; A61B 5/145; A61B 5/0205

(52) U.S. Cl. CPC A61B 5/4836; A61B 5/7282; A61B 5/4818; A61B 5/14542; A61B 5/0205; A61B 5/0476; A61B 5/0488

(57) ABSTRACT

The invention relates to an active implantable medical device. The device measures for respiratory activity and for detection of a ventilation rate fall event below a predetermined threshold. The device analyzes in real time the various parameters of the ventilation rate fall event and determines whether they satisfy predefined criteria, so as to allocate or not a priori hypopnea suspicion indicator to the event. An anti-hypopnea therapy is selectively triggered on detection of respiratory collapse, but only in the case of current events indicating a priori hypopnea suspicion. The criteria may include: history of intervals between successive events constituting hypopnea episodes; severity of the current event; conformity of a current event profile with a reference profile; physiological, obstructive or central, origin of the event; patient's current sleep stage; and history of the degree of efficacy of the therapies.

### CROSS-REFERENCE TO RELATED PATENT APPLICATIONS

This application claims the benefit of and priority to French Patent Application No. 1460320, filed on Oct. 27, 2014, which is incorporated herein by reference in its entirety.

### BACKGROUND

The invention relates to the diagnosis and treatment of sleep disordered breathing, particularly those related to a condition known as sleep apnea syndrome (SAS).

Sleep apnea syndrome (SAS) or more precisely sleep apnea-hypopnea syndrome (SAHS) is manifested by stops (apneas) and/or frequent reductions (hypopneas) of the respiratory flow during the night (at least 10-20 times per hour). Hypopneas, like apneas, have significant effects on the patient's physiological balance, such as the risk of hypoxemia, arousal, etc.

The two characteristic disorders of this disease should be distinguished, namely apnea (or breathing pauses), defined as temporary cessation of respiratory function longer than 10 s, occurring in a patient's sleep phase; and hypopneas, defined as significant decay, without interruption, of respiratory rate during a patient's sleep phase.

The present invention relates to the diagnosis and treatment of hypopnea.

Hypopnea is essentially characterized by:

i) a significant reduction in the amplitude of the respiratory rate over a period of at least 10 s, and
ii) a subsequent oxygen desaturation of at least 3 to 4% and/or consecutive arousal.

More specifically, the respiratory rate reduction threshold is generally set at 30% for a subsequent desaturation of 4% or 50% of the basic respiratory amplitude for a subsequent desaturation of 3% (AASM Manual for The Scoring of Sleep and Associated Events, 2007). It is noted that these threshold values are only typical values, and may vary from one patient to another and, for the same patient, can change over time, either during a single night or from one night to the next.

In this description, the term “hypopnea” is used only to describe a reduction of ventilatory rate followed by oxygen desaturation or arousal (official definition of hypopnea according to the American Academy of Sleep Medicine, published in the Journal of Clinical Sleep Medicine, volume 8, No. 5, 2012).

When hypopnea is not proven but only suspected, because of early detection only on a criterion of airflow limitation, the terms “ventilatory drop”, “drop in respiratory amplitude”, or “ventilatory reduction (of the flow)” are used.

Hypopneas are the most common respiratory disorder in patients subject to SAS. Thus, a patient with severe SAS can have during a single night one apnea but up to 450 hypopneas.

There are various techniques known for treating hypopneas by various stimulations that open the airways (in case of obstructive respiratory disorders) or stimulate the nervous centers that control breathing (in case of central respiratory disorders).

To do this, it is essential to detect hypopnea automatically and in real time from its appearance, with the difficulty that the detection of a ventilation fall is not sufficient to prove hypopnea. Indeed, one can be sure that the event was indeed a hypopnea when this event is completed (establishing then that it fulfills the duration, oxygen desaturation or arousal onset).

The real-time detection of apnea may be relatively easy relative to that of hypopneas. Indeed, the criteria for defining a hypopnea include oxygen desaturation and arousal, which both occur after the hypopnea. This occurs a long time after the detection of the respiratory drop (typically between 10 and 60 seconds after), that is to say, when normal breathing is restored. It is thus too late to treat hypopnea because it has ended.

It is certainly possible to treat preventively, without delay, all the events of ventilatory drop, e.g. 10 s after onset, without waiting for confirmation that it is indeed a hypopnea (which involves desaturation or arousal detection). However, all ventilatory falls are not necessarily hypopnea events and, specifically, the current real-time sensors have a positive predictive value that does not exceed 60-70%. In other words, for a patient with severe SAS, which can have 400 hypopneas per night, such a detector will see 570 events and the patient will be treated unnecessarily 170 times in the night. This overtreatment presents the double risk of disrupting patient sleep and of promoting the habituation effect, the therapies losing their effectiveness over time. These effects and consequences would invalidate the beneficial effects of treatment, and could eventually lead to poor compliance.

To avoid this risk, it has been proposed to treat only certain events, or adapt the type of therapy to the type of apnea or hypopnea detected:

EP 1336422 A1 (ELA Medical) proposes to detect an apnea or a hypopnea from a ventilatory signal obtained by an implantable pacemaker. If certain cardiac hemodynamic conditions exceed a reference threshold, then one or more pacing parameters are changed, such as pacing rate, atrioventricular delay, etc.;

US 2003/0153956 A1 also proposes to detect an apnea or a hypopnea from a ventilatory signal picked up by an implantable pacemaker. To stop the breathing disorder, the base frequency is adjusted, but the adjustment occurs only after detecting a minimum number of proven respiratory disorders; and

U.S. Pat. No. 7,371,220 B1 proposes to detect an apnea or hypopnea event by an implantable device, in real time, at the onset of ventilatory fall. A differentiated therapy is applied according to the type of event (obstructive or central origin), but there is no provision for selection of the events to be treated, (i.e., all events, whether of one type or another, are the subject of the therapy).

None of these techniques has been proved fully satisfactory to improve the treatment of sleep disordered breathing, as they do not solely target hypopnea events. In particular, none of these techniques can treat a maximum of hypopneas in real time, without the risk of compromising the effectiveness of the overall treatment by including therapies triggered by detection of ventilatory falls that do not correspond, in fine, to proven hypopneas.

U.S. Pat. No. 7,942,824 B1 and US 2010/307500 A1 are specifically related to the detection and treatment of hypopnea, but they plan to apply a therapy only in cases of confirmed hypopnea. The techniques proposed in these documents do not solve the problem mentioned above, that the mere detection of a ventilatory fall (which may not be a harbinger of a hypopnea) is not sufficient in itself to prove the occurrence a hypopnea, with the disadvantage that the therapy is applied only relatively late, that is to say after hypopnea has completely developed its deleterious effects.

### SUMMARY

Various embodiments of the invention provide a very different approach from those presented so far, and are intended to automatically make an analysis and classification of ventilatory falls, detected in real time, to better target the most severe or the most certain hypopneas, and apply therapy only to the latter.

The advantage of such a solution is not only to prevent over-stimulation of the patient, but also of being able, if a therapy is to be applied, to adjust the parameters of this therapy according to the type of hypopnea.

More specifically, the invention proposes a medical device of the general type disclosed by the U.S. Pat. No. 7,942,824 B1 cited above, the medical device including a processor configured to measure respiratory activity, the measurement being a representative value of the instantaneous ventilatory flow rate of a patient; detect a ventilatory fall event, able to detect in real time a decrease in the flow rate value below a predetermined threshold; and provide anti-hypopnea therapy.

According to an exemplary embodiment, the processor of the device is further configured to discriminate, during said ventilatory fall event and in real time, at least one parameter of the current ventilatory fall event and determine if this parameter satisfies at least one predefined criterion for a priori assigning the event an hypopnea suspicion indicator or an hypopnea non-suspicion indicator; and selectively apply at least one anti-hypopnea therapy, before the end of said ventilatory fall event, able to trigger from the crossing of said predetermined threshold, an anti-hypopnea therapy only in case of attribution to the current event of a priori hypopnea suspicion indicator.

According to various advantageous subsidiary characteristics:

the device further inhibits any anti-hypopnea therapy, in case of attribution to the current event of a priori hypopnea non-suspicion indicator;

the at least one predefined criterion includes the value of a counter of successive ventilatory fall events detected in the interval of a predetermined period corresponding to the duration of an hypopnea episode, a hypopnea suspicion indicator is assigned to the current event only if the counter value exceeds a predetermined minimum value, and the counter resets if the elapsed time between two successive ventilatory fall events exceeds said predetermined period corresponding to the duration of an hypopnea episode;

the device further measures a physiological signal as a function of the respiratory activity, detects of the crossing of a limit threshold by the physiological signal, and forces the increment of the counter at any detection of a ventilatory fall event;

the predetermined period and/or the predetermined minimum value are variable adaptive values, and the device further automatically calculates the predetermined period and/or of the predetermined minimum value, respectively, during the night;

the device further measures a physiological signal as a function of the respiratory activity, detects the crossing of a limit threshold by a respiratory signal or by the physiological signal, and forces the triggering the anti-hypopnea therapy from the crossing of said limit threshold;

the physiological signal is a signal of the group formed by: blood oxygen saturation rate, blood oxygen desaturation rate, heart rate, blood pressure, pulse wave, EEG, electromyogram and combinations thereof;

the at least one predefined criterion includes a hypopnea reference profile stored in memory, the device further determines a current profile for the ventilatory fall event and evaluates a correlation between the current profile and the reference profile, and the discrimination includes assigning a suspicion hypopnea indicator to the current event if the correlation between the current profile and the reference profile exceeds a minimum predetermined correlation value;

the device further is adapted to determine or not, after the end of the suspected hypopnea event, the presence of a hypopnea consecutive to the detection of a ventilatory fall event, and is capable of updating the hypopnea reference profile stored in memory, in case of proven hypopnea;

the discrimination includes analyzing a plurality of parameters of the current ventilatory fall event according to a plurality of predefined criteria, assigning, for the current ventilatory fall event, a respective specific score to each of the predefined criteria, establishing an overall composite score based on a combination of the specific scores, and assigning to the current event a priori hypopnea suspicion indicator or a priori hypopnea non-suspicion indicator based on the result of a comparison of the global score to a predetermined score threshold;

the discrimination further includes adapting to select depending on the value of the global score a particular therapy among several possible therapies; and

the at least one predefined criterion is a criterion of the group formed by: historical of intervals between successive ventilatory fall events constituting hypopnea episodes; severity of the current ventilatory fall event; conformity of a profile of the current ventilatory fall event with a reference profile; physiological, obstructive or central, origin of the ventilatory fall event; patient's current sleep stage; and history of the degree of efficacy of the applied therapies.

### BRIEF DESCRIPTION OF THE DRAWINGS

Further features, characteristics and advantages of the present invention will become apparent to a person of ordinary skill in the art from the following detailed description of preferred embodiments of the present invention, made with reference to the drawings annexed, in which like reference characters refer to like elements and in which:

FIG. 1 schematically illustrates the various components of a device for one implementation of the invention.

FIGS. 2a and 2b are histograms detailing clinical records for two different patients showing the distribution of episodes (intervals between successive hypopneas during a sleep night).

FIG. 3 is a diagram illustrating a first embodiment of the invention, by a technique based on the analysis of the temporal sequence of hypopnea events to decide to trigger, or not, therapy in case of occurrence of ventilatory fall.

FIG. 4 is a diagram detailing the steps implemented by the technique illustrated in its principle in FIG. 3.

FIG. 5 is a diagram illustrating another example of implementation of the invention, a technique based on the analysis of the detected ventilatory fall profile and its comparison with a reference profile to decide to trigger, or not, therapy in case of occurrence of ventilatory fall.

FIG. 6 is a flow diagram showing yet another embodiment of the invention, based on a multi-criteria technique wherein the ventilatory fall is analyzed in real time and simultaneously, facing a plurality of criteria, giving respective indexes that will be weighted and combined to decide to trigger, or not, a therapy in case of occurrence of ventilatory fall.

### DETAILED DESCRIPTION

An exemplary embodiment of the invention will now be described.

Regarding its software aspects, the invention may be implemented by appropriate programming of the controlling software of a known, external or implantable, active medical device, provided with therapy functions of sleep disordered breathing.

These devices include a programmable microprocessor provided with circuits for shaping and delivering stimulation pulses to implanted electrodes. It is possible to transmit to it by telemetry software that will be stored in memory and executed to implement the functions of the invention which will be described below. The adaptation of these devices to implement the functions of the invention is within the reach of a skilled-in-the-art person and will not be described in detail.

The method of the invention is implemented primarily by software, through appropriate algorithms performed by a microcontroller or a digital signal processor. For the sake of clarity, the various processing applied will be decomposed and schematized by a number of separate functional blocks in the form of interconnected circuits, but this representation, however, is only illustrative, these circuits including common elements in practice corresponding to a plurality of functions generally performed by the same software.

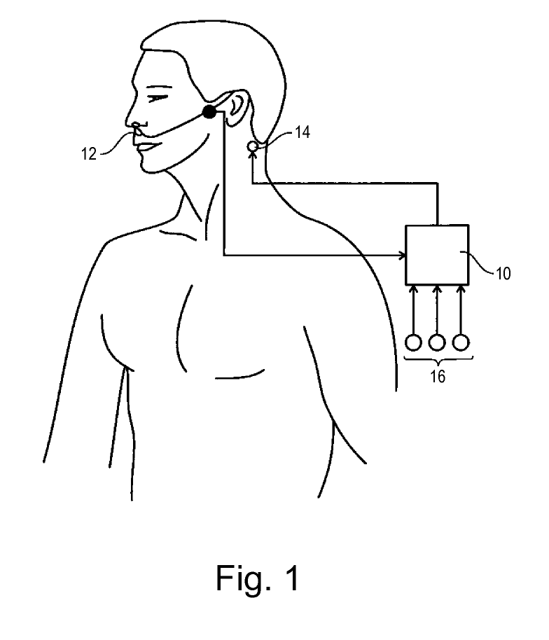

FIG. 1 schematically illustrates the main elements necessary for the implementation of the invention.

This implementation is carried out by a device 10, which can be an external device, such as a Holter recorder connected to various sensors or electrodes (or an implantable device, such as a pacemaker, resynchronizer and/or defibrillator).

The device 10 is configured to measure the respiratory flow. In the case of an external device, it may be for example a nasal pressure cannula 12 (and/or an oral cannula) or other type of sensor such as a thermistor or sensor of mechanical changes in volumes of the abdomen and/or of the chest (by a belt equipped with sensors sensitive to stretching). In the case of an implantable device, the measuring of the respiratory flow includes measuring the transthoracic impedance between the implant housing and a remote electrode.

The device 10 is further connected to a device for applying an anti-hypopnea therapy. It may include, but in no way be limited to, a kinesthetic effector 14, constituted for example by a vibrator placed in a sensitive region of the skin, typically (in adults) in the region of mastoid bone close to the ear. Vibrotactile stimulation applied to the skin by the effector 14 is detected by the sensory receptors or mechanoreceptors in the body, and transmitted through sensory nerves to the autonomous central nervous system.

The device 10 is also connected to various sensors 16 such as electrodes, optical fibers, accelerometers, microphones, etc. for measuring physiological signals such as heart rate, oxygen saturation (measured by a sensor placed on the finger or on the ear), the phonocardiogram, the electroencephalography or the electromyography. This provides, besides the measurement of respiratory flow, additional signals which allow on one hand to better distinguish the ventilatory falls herald of hypopneas, and on the other hand, to determine a posteriori if the event detected at the origin of ventilatory fall was or was not a hypopnea.

Various embodiments of the invention specifically target the diagnosis and treatment of hypopnea (and not of the apneas), especially following the early detection of ventilatory fall, and without waiting for the end of the event (thus without knowing a priori whether it is or is not a true hypopnea).

The purpose is notably to discriminate between a ventilatory fall harbinger of a real hypopnea (in which case, it is immediately treatable by therapy) or not, in which case it will be necessary not to apply any therapy to prevent the development of unnecessary adverse effects such as cough, arousal, etc.

Various therapies that can be applied to a proven hypopnea are known in the art. Various embodiments of the invention determine whether to trigger (or not trigger) the application of a therapy at an advanced stage of a suspected hypopnea (suspicion coming from the detection of ventilatory fall), optionally by operating a selection between several possible therapies, but without changing the mode of application of this therapy.

For reference, in the case of an external device, it may be a kinesthetic stimulation therapy, for example as described in WO 2007/141345 A1 (FR 2908624 A1). In the case of an implantable device, it can be a therapy by modification of the pacing rate and/or of the atrioventricular delay.

According to various embodiments of the invention, once a ventilatory fall is detected, it is considered that there is hypopnea suspicion (hypopnea which can be proved only after the end of the event, so very late) and then an analysis to decide early is operated. The analysis is conducted without waiting for the end of the event, of the trigger or not of an anti-hypopnea therapy and to refrain from any therapy for events wherein ventilatory fall is probably not the harbinger of a hypopnea.

This analysis, to determine the trigger or not of a therapy can be based on several, alternative or cumulative, strategies corresponding to various diagnostic criteria that will be described in detail below.

Criterion 1: Succession in Episodes of Hypopnea Events

Polysomnography diagnostics show that the hypopnea events occur by “episodes”. The events occur in a roughly regular succession within relatively short temporal intervals, the different episodes being separated by longer breaks. A first criterion is to assess whether the detected ventilatory fall corresponds to such an “episode”, in order to apply a therapy only in this case, so as to treat a maximum hypopnea while limiting the risk of unnecessary therapies which could have deleterious effects.

It is therefore necessary to determine whether the detected ventilatory fall is isolated in time, or whether it is part of an “episode”.

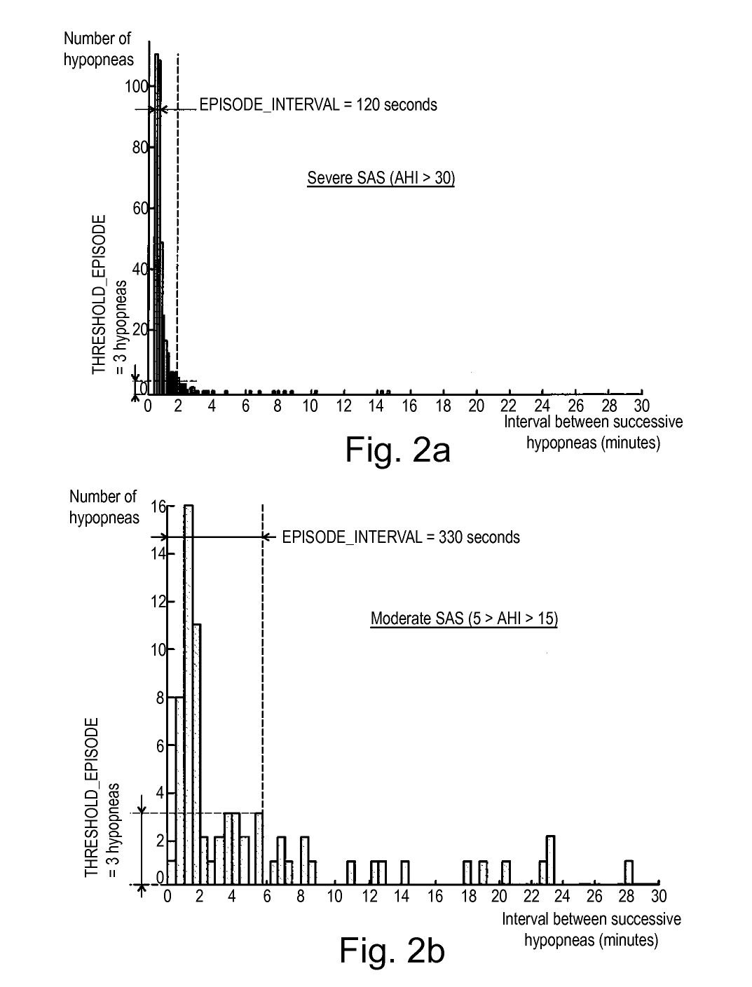

The phenomenon of episodes however presents a great variability from one patient to another, as shown in FIGS. 2a and 2b. FIGS. 2a and 2b are histograms of clinical records detailing for two respective different patients the distribution of episodes (the intervals between successive hypopneas during a night's sleep).

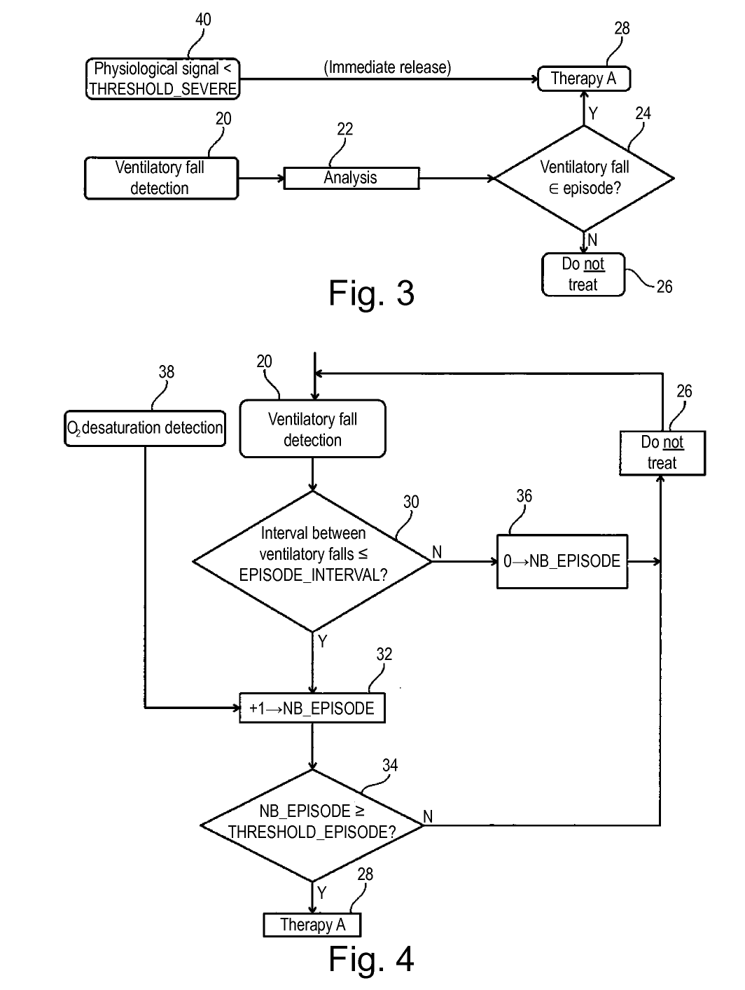

The corresponding analysis method is illustrated in FIGS. 3 and 4.

As shown in FIG. 3, upon detection of a ventilatory fall (block 20), the ventilatory fall is analyzed (block 22). If it is considered as not belonging to an episode (block 24), no therapy is applied (block 26). Otherwise, a predetermined anti-hypopnea therapy (“Therapy A”) is applied (block 28).

More specifically, as shown in FIG. 4, on detection of a ventilatory fall (block 20), the method estimates the time elapsed since the last ventilatory fall, and verifies that the temporal interval between these two successive ventilatory falls does not exceed a predetermined time interval EPISODE_INTERVAL (block 30). If this is the case, the ventilatory fall is considered to belong to a hypopnea episode and a counter NB_EPISODE of the number of hypopneas in the episode is incremented (block 32). In case apnea (which may be regarded as an extreme form of hypopnea) is detected during the EPISODE_INTERVAL temporal interval, then the counter NB_EPISODE is also incremented (while apnea may be treated as apnea from its appearance).

The value of the counter NB_EPISODE is then compared to a threshold THRESHOLD_EPISODE (block 34), corresponding to a minimum confidence level, and if this is the case, therapy is initiated (block 28).

If at block 30 it is found that the interval between the ventilatory fall that has just been detected from the preceding one exceeds the value EPISODE_INTERVAL, then the hypopnea counter NB_EPISODE is reset (block 36). The isolated ventilatory fall will not be subject to therapy (block 26).

Note that EPISODE_INTERVAL and THRESHOLD_EPISODE values can be adapted to the patient by a setting following for example a diagnostic polysomnography, prior to the implementation of the automatic system. Thus, in the case of a patient with severe SAS (apnea-hypopnea index AHI>30) EPISODE_INTERVAL may be fixed at 120 s and THRESHOLD_EPISODE to 3 hypopneas. For a patient with moderate SAS (5<AHI<15) EPISODE_INTERVAL can be fixed to 330 s, and THRESHOLD_EPISODE to 3 hypopneas.

Note also that these two parameters may change for the same patient who, if he/she is responder to the treatment, may be subjected to an increase in EPISODE_INTERVAL and/or in THRESHOLD_EPISODE. This adaptation over time can be made during an automatic or manual analysis, of the results of the detection of hypopneas stored before morning and searchable by a doctor. It is also possible, if the doctor finds too great a discrepancy in the number of ventilatory falls that triggered therapy compared to the number of proven hypopneas (that is to say with desaturation and/or arousal at the end of hypopnea), to adjust these settings.

In an implementation variant, the EPISODE_INTERVAL and/or THRESHOLD_EPISODE values are automatically recalculated during the night. For example, the statistical properties of the detected and proven events can be analyzed. The intervals between consecutive hypopneas, which are likely to follow a Poisson distribution (FIG. 2a) that can be set, can be updated gradually during the night to define an adaptive EPISODE_INTERVAL value from an analytical formulation of the statistical law.

If the patient is equipped with a sensor for detecting oxygen desaturation (typically a decrease of at least 3% of oxygen saturation as compared to the base saturation), it is possible to use this information, which is a criterion for confirmation of hypopnea, to improve the functioning of the analysis algorithm of hypopnea episodes.

Indeed, the detection of oxygen desaturation that occurs after hypopnea and therefore between two successive hypopneas, confirms that the previous ventilatory fall was effectively heralding hypopnea, which reinforces the need for therapy. Accordingly, especially for a patient with severe SAS, it is possible to incorporate an additional criterion for the detection of an episode, an episode being possibly defined, for example, as consisting of three repeating hypopneas separated by less than 120 s, or as two hypopneas repeated in less than 120 s, if there have been during the 120 s detection of oxygen desaturation. For this, as shown in FIG. 4, the detection of oxygen desaturation (block 38) causes the increment of the counter NB_EPISODE (block 32), regardless of the detection of ventilatory fall.

Note that this case is not limited to the oxygen desaturation. For example, if the sensor used is of the type EEG, it may be a micro-arousal, and any micro-arousal detected between two successive hypopneas of the same episode also increments the counter NB_EPISODE.

Criterion 2: Severity of the Event Related to the Detected Ventilatory Fall

According to this second criterion, the analysis set forth above (which is intended to determine whether a hypopnea episode is occurring) is bypassed so as to cause the immediate trigger and without another condition of therapy if a physiological signal falls below a predetermined severity threshold THRESHOLD_SEVERE (block 40, FIG. 3). In this case, ventilatory fall is immediately processed (block 28), without waiting for confirmation of the analysis in episodes. For example, if blood oxygen saturation is detected below 90% of the base saturation, this measure is a sign of severe disease requiring immediate therapy.

Similarly, in case of a very significant reduction in ventilation rate, or even in case of a reduction that degenerates in apnea, then immediate therapy will be applied to the event.

Another possible example of a physiological signal is the heart rate. If it slows down below a threshold, then therapy is immediately applied.

In all cases, the threshold is a parameter initially determined by the physician but may be changed automatically or manually, depending on the disease progression and on the treatment efficacy.

Note also that this severity criterion has priority over the other criteria described below, so that if this criterion is satisfied, then therapy will be triggered immediately upon detection of ventilatory fall.

Criterion 3: Characteristic Profile of the Event

It is generally seen that the hypopneas are stereotypically repeated during the same night, with an amplitude, duration, morphology, etc., constituting a characteristic “profile” of a same patient. Therefore, it is possible to use a criterion based on the analysis of the hypopnea profile, to trigger therapy only if the ventilatory fall matches the characteristic profile recorded for the patient, while the new, unrecognized, types of profiles will not be subject to therapy. These new profiles are however stored to increase the number of stored profiles, and will then be considered as known profiles that, if repeated, may in turn form a triggering criterion of therapy.

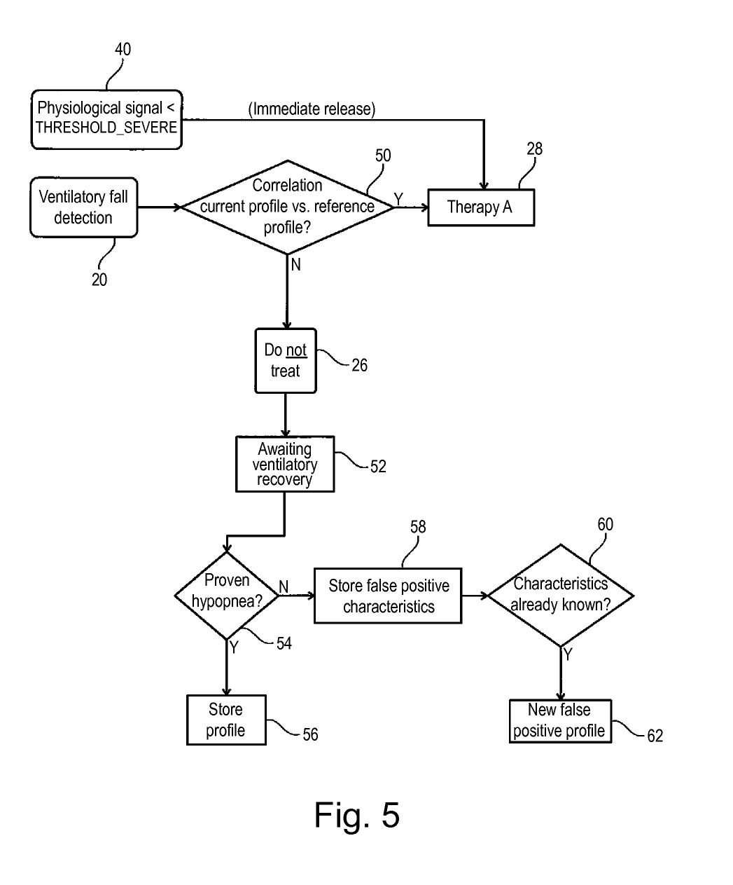

FIG. 5 illustrates in more detail the method to implement this criterion based on the profile analysis.

Several hypopneas profile aspects may be identified in a patient, according to:

The amplitude of the ventilatory fall;

The morphology of the respiratory cycles with ventilatory fall, these cycles having a more flattened and squared shape, while the normal cycles have a rounder shape;

The sudden or gradual speed of the ventilatory fall, and the fact that it is or not preceded by hyperpnoea;

The duration of ventilatory fall;

The presence or absence of hyperpnoeas between hypopneas and apneas.

A number of profiles corresponding to these features are stored in a memory of the device.

Once a ventilatory fall is detected (step 20), the device checks (block 50) whether or not its characteristics correspond to one of the saved profiles, for example by calculating a correlation function between the current profile and the reference profile and checking if the degree of correlation exceeds a given threshold. If so, therapy is applied (block 28).

If a ventilatory fall is detected but does not correspond to one of the saved profiles, no therapy is applied (block 26). The device then waits for the end of the event (block 52) and determines, based on the desaturation and/or arousal criteria, whether or not the event was hypopnea (block 54):

In the affirmative (meaning that hypopnea was not suspected despite detected ventilatory fall), then a new reference profile is created and stored in the device memory (block 56) so as to improve the efficiency of the device;

Otherwise (the ventilatory fall was not heralding hypopnea), the profile is stored in memory (block 58) and if this type of event occurs again (block 60), a false positive profile is created and saved (block 62), this information being possibly used later by the practitioner to reconfigure the device to improve specificity.

In this example too, the event severity criterion can be activated, giving it a higher priority than the profile analysis criteria so that the detection of the crossing of the severity threshold (block 40) causes the immediate therapy delivery (block 28).

Criterion 4: Physiological Origin of Hypopnea

Just as apneas, hypopneas can be of two origins:

Obstructive, when hypopnea is due to airway obstruction, probably caused by muscle weakness;

Central, when hypopnea is caused by a control fault of respiration in the central nervous system.

The classification between obstructive hypopneas and central hypopneas can be used as a criterion for deciding whether or not to apply a therapy.

Indeed, in some patients a high prevalence of obstructive events is found, while others have a strong predominance of central events. Therefore, the occurrence of ventilatory fall having the characteristics of a non-predominant event may be due to a false detection, and in this case it will be chosen not to apply therapy. For example, if a patient is considered making obstructive hypopneas in 90% of cases, the detection of a ventilatory fall, typical of central hypopnea, will not trigger therapy.

The determination of the origin of the hypopnea may be performed in real time by a profile analysis of the same type as what was described above for the criterion No. 3, in particular on the basis of the following characteristics (one can in particular refer to the article of Renderath et al published in Sleep, 2013; 36 (3): 363-368):

Morphology of respiratory cycles: in case of hypopneas, a crushed form of the inspiration curve reveals an obstructive origin;

Paradoxical breathing: obstructive hypopnea may cause a phase shift between the chest respiratory movements and those of the abdomen;

Ventilation recovery: gradual for a central hypopnea, sudden for obstructive hypopnea;

Micro-arousal sequencing: they appear at the beginning of the ventilation recovery in the case of an obstructive hypopnea, and at the maximum of the ventilation recovery in the case of central hypopnea;

Sleep stage of after micro-arousal: REM sleep is more conducive to obstructive hypopneas.

The classification between obstructive and central hypopnea can be used also as a criterion to differentiate the applied therapy.

Thus, in the case of central hypopnea, it is preferable to deliver a therapy activating the diaphragm, for example by stimulation of the phrenic nerve, whereas for obstructive hypopnea it is preferable to stimulate the upper airway muscles, e.g. via the hypoglossal nerve or the laryngeal nerve.

Criterion 5: Sleep Stage

The wakefulness or sleep state, and the different sleep stages, affect the respiratory rhythm.

In particular, during periods of wake or of sleep, or even of REM sleep, breathing is not stable, and sudden and significant, but not pathological, variations of periods and amplitudes of respiratory cycles can be observed.

Therefore, changes in respiratory rate and amplitude may not be related to hypopnea and it will be preferable in this case to suspend the application of a therapy.

In contrast, ventilation falls occurring in slow wave sleep can be processed with a higher priority, the ventilation fall in this case being with a high probability heralding hypopnea.

Criterion 6: Treatment Efficacy

When a number of suspected hypopneas (detected ventilatory falls) do not respond to anti-hypopnea treatment during the night (or successive nights), this rate reduction typology must be stored for later analysis. If it turns out a posteriori that these are not proven hypopneas, then they shall no longer be detected (false positive as in the case of criterion 3). If however, it turns out that these are proven hypopneas, then an alert can be issued in order to inform the physician that initially applied therapies may no longer be appropriate. In all cases, the treatment of this type of ventilatory falls should be suspended.

Combined Use of Criteria No. 1-6

The criteria that have been described above can be used together to make the decision whether to apply or not a therapy upon detection of a ventilatory fall.

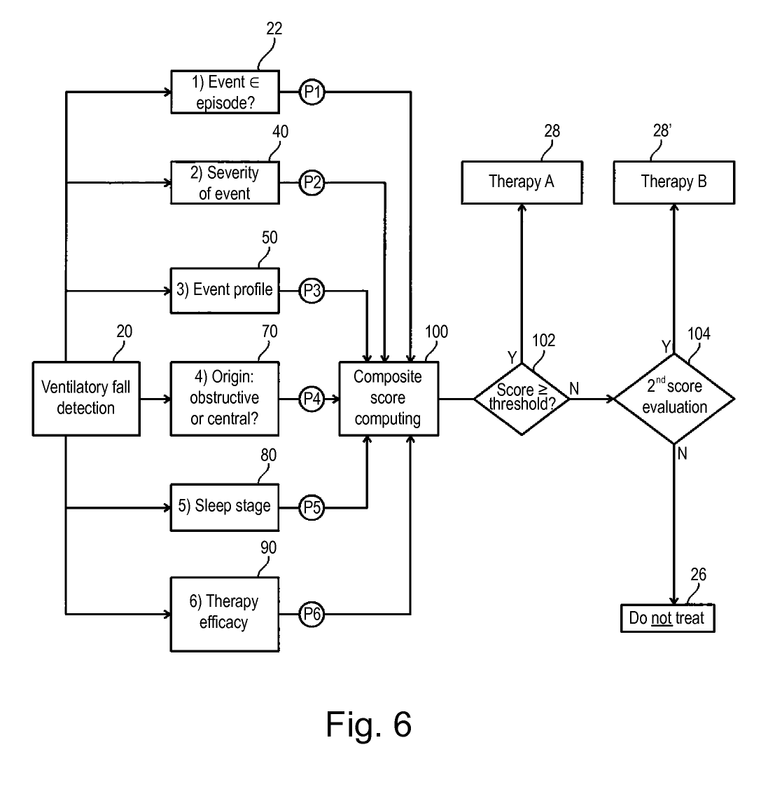

As shown in FIG. 6, upon detection of a ventilatory fall (block 20) different criteria are evaluated together (blocks 22, 40, 50, 70, 80, 90).

Each criterion is allocated a weighting P1 to P6, for example, by assigning to each of the criteria a number of positive or negative points constituting an elementary score. For example, the elementary scores P1 to P6 respectively corresponding to the criteria No. 1 to No. 6 discussed above can take the following values:

P1=value of the counter NB_EPISODE (described in FIG. 4, incremented in step 32);

P2=+5 if THRESHOLD_SEVERE is crossed, 0 if the current profile is unknown, +3 if the current profile corresponds to a known reference profile;

P4 (multiplicative factor)=+1 for a predominant central origin or by default of unknown origin, −1 for predominant obstructive origin;

P5=−2 during REM sleep, −5 during wake, +1 in slow wave sleep;

P6=−3 if the patient is unresponsive to treatment, +2 if the patient is responding to treatment, 0 if unspecified response.

A composite score is calculated (block 100) by addition and/or multiplication of points P1 to P6.

In the example above, the composite score is calculated by adding the individual scores P1 to P3 and P5 to P6, and multiplication by the individual score P4:

S=(P1+P2+P3+P5+P6)×P4.

The score is then compared to a predetermined threshold (block 102), for example a threshold equal to 5 with the values of the previous example, and if this threshold is crossed a predetermined therapy is applied (“Therapy A”, block 28).

Otherwise, and if several therapies are available (in particular for differentiated treatment of obstructive hypopneas and of central hypopneas), a second evaluation score is made (block 104) and according to the result a different therapy (“Therapy B”, block 28′) is applied, or it is decided not to apply any therapy to the patient (block 26). With the values of the previous example, Therapy B is applied if the score is negative but if its absolute value exceeds the threshold, no therapy is applied in the opposite case.

This combination of criteria allows to take into account the case of complex cases and to selectively trigger therapy in very specific situations, for example:

Hypopnea with known profile, occurring in slow wave sleep;

Second hypopnea of an episode, for a responder patient, in slow wave sleep;

Hypopnea of known profile, in slow wave sleep, for a responder patient;

What is claimed is:

1. An active medical device, comprising:
a processor configured to:
measure respiratory activity, the measurement providing a representative value of the instantaneous ventilatory flow rate of a patient;
detect a ventilatory fall event by detecting in real time a decrease in the value of the instantaneous ventilatory flow rate below a predetermined threshold; and
provide anti-hypopnea therapy, wherein the processor is configured to:
assess at least one parameter of the ventilatory fall event, the processor adapted to analyze in real time during the ventilatory fall event and determine if the parameter satisfies at least one predetermined criterion, for assigning to the ventilatory fall event a hypopnea suspicion indicator or a non-hypopnea suspicion indicator; and
selectively applying an anti-hypopnea therapy in the case of attribution to the current ventilatory fall event the hypopnea suspicion indicator, the anti-hypopnea therapy triggered before the end of the ventilatory fall event.

2. The device of claim 1, wherein the processor is further configured to:
inhibit the anti-hypopnea therapy in case of attribution to the current ventilatory fall event of the non-hypopnea suspicion indicator.

3. The device of claim 1, wherein:
the at least one predefined criterion comprises the value of a counter of successive ventilatory fall events detected within the range of a predetermined period corresponding to the duration of an episode of hypopneas;
assessing the at least one parameter further comprises assigning a suspicion indicator of hypopnea to the current ventilatory fall event only if the counter value exceeds a minimum predetermined value; and
the counter is reset if the time between successive ventilatory fall events exceeds the predetermined period corresponding to the duration of an episode of hypopneas.

4. The device of claim 3, wherein the processor is further configured to:
measure a physiological signal as a function of the respiratory activity;
detect the crossing of a threshold limit by the physiological signal; and
increment the counter at any detection of a ventilatory fall event.

5. The device of claim 3, wherein the predetermined period and/or the predetermined minimum value are variable adaptive values, and wherein the processor is further configured to:
calculate, respectively, the predetermined period and/or the predetermined minimum value during the night.

6. The device of claim 1, wherein the processor is further configured to:
measure a physiological signal as a function of the respiratory activity;
detect the crossing of a threshold limit by a respiratory signal or by the physiological signal; and
trigger the anti-hypopnea therapy at the crossing of the limit threshold.

7. The device of claim 6, wherein the physiological signal is at least one of a level of blood oxygen saturation, a blood oxygen desaturation rate, a heart rate, a blood pressure, a pulse wave, an electroencephalogram, an electromyogram or any combination thereof.

8. The device of claim 1, wherein:
the at least one predefined criterion comprises a reference profile of hypopnea stored in memory;
the processor is further configured to determine a current profile for the current ventilatory fall event and for evaluate a correlation between the current profile and the reference profile; and
the processor is further configured to assign a hypopnea suspicion indicator to the current event if the correlation between the current profile and the reference profile exceeds a predetermined correlation minimum value.

9. The device of claim 8, wherein the processor is further configured to:
determine, after the end of the suspected hypopnea event, the presence of an hypopnea consecutive to the detection of the ventilatory fall event; and
update the hypopnea reference profile stored in memory, in case of a determined hypopnea.

10. The device of claim 1, wherein the discrimination of at least one parameter of the current ventilatory fall event comprises:
analyzing a plurality of parameters of the current ventilatory fall event according to a plurality of predefined criteria;
assigning, for the current ventilatory fall event, a respective specific score to each of the predefined criteria;
determining an overall composite score based on a combination of specific scores; and
assigning to the current event suspicion an a priori suspicion indicator of hypopnea or non-hypopnea based on the result of a comparison of the overall score to a predetermined score threshold.

11. The device of claim 10, wherein the discrimination of at least one parameter of the current ventilatory fall event comprises:
selecting, depending on the value of the global score, a particular therapy among several possible therapies.

12. The device of claim 1, wherein the at least one predefined criterion is at least one of a history of intervals between successive events constituting the ventilatory fall hypopnea episodes; a severity of the current ventilatory fall event; a conformity of a profile of the current ventilatory fall event with a reference profile; a physiological origin, obstructive or central, of the ventilatory fall event; the patient's current sleep stage; and a history of the degree of efficacy of the applied therapies.
